(19) United States
(12) Patent Application Publication
(10) Pub. No.: US 2025/0195201 A1
(43) Pub. Date: Jun. 19, 2025

(54) ARTIFICIAL BLOOD VESSEL

(71) Applicant: HI-LEX CORPORATION, Hyogo (JP)

(72) Inventor: Shinsaku KOARASHI, Hyogo (JP)

(21) Appl. No.: 18/849,727

(22) PCT Filed: Mar. 29, 2023
(86) PCT No.: PCT/JP2023/012804

§ 371 Date: Sep. 23, 2024

(30) Foreign Application Priority Data
Apr. 7, 2022 (JP) 2022-064118

(51) Int. Cl. A61F 2/06; A61F 2/00; A61L 27/40; A61L 27/50; D03D 13/00

(52) U.S. Cl. CPC A61F 2/06; A61L 27/40; A61L 27/507; D03D 13/004; A61F 2002/0081; D10B 2403/01; D10B 2509/06

(57) ABSTRACT

The artificial blood vessel of the present invention is an artificial blood vessel having a predetermined weaving structure in which a warp yarn and a weft yarn are woven, wherein at least one of the warp yarn and the weft yarn is composed of a multifilament yarn including a plurality of filament yarns, and wherein the artificial blood vessel has a plurality of covering parts provided at a plurality of locations on a surface of the artificial blood vessel, each of the plurality of covering parts covering the multifilament yarn in a planar shape, and uncovered parts that are provided between the plurality of covering parts and are not covered by the covering parts on the surface of the artificial blood vessel.

### TECHNICAL FIELD

The present invention relates to an artificial blood vessel.

### BACKGROUND ART

The artificial blood vessel is used, for example, for replacing a pathological living blood vessel. The artificial blood vessel is composed of a weaving structure of a warp yarn and a weft yarn, as shown in, for example, Patent Document 1. The artificial blood vessel is required to have little blood leakage from the artificial blood vessel, that is, a high blood leakage resistance. The artificial blood vessel can improve in blood leakage resistance by increasing weave densities of the warp yarn and the weft yarn.

### PRIOR ART DOCUMENT

### Patent Document

Patent Document 1: JP 2012-139498 A

### SUMMARY OF THE INVENTION

### Problems to Be Solved by the Invention

However, although blood leakage resistance can be improved by increasing the weave density of the artificial blood vessel, flexibility required for the artificial blood vessel can be impaired.

Therefore, it is an object of the present invention to provide an artificial blood vessel capable of improving blood leakage resistance while maintaining flexibility.

### Means to Solve the Problem

The artificial blood vessel of the present invention is an artificial blood vessel having a predetermined weaving structure in which a warp yarn and a weft yarn are woven, wherein at least one of the warp yarn and the weft yarn is composed of a multifilament yarn including a plurality of filament yarns, and wherein the artificial blood vessel has a plurality of covering parts provided at a plurality of locations on a surface of the artificial blood vessel, each of the plurality of covering parts covering the multifilament yarn in a planar shape, and uncovered parts that are provided between the plurality of covering parts and are not covered by the covering parts on the surface of the artificial blood vessel.

### Effects of the Invention

According to the artificial blood vessel of the present invention, blood leakage resistance can be improved while maintaining flexibility.

### BRIEF DESCRIPTION OF THE DRAWINGS

FIG. 1 is a side view of an artificial blood vessel according to one embodiment of the present invention.

FIG. 2 is a partially enlarged view of a region II in FIG. 1.

FIG. 3 is a fabric structure diagram showing one example of a weaving structure of a base material used in the artificial blood vessel of FIG. 1.

FIG. 4 is a schematic view of a cross section of the base material taken along line IV-IV in FIG. 3.

FIG. 5 is a schematic view of a cross section of the base material taken along line V-V in FIG. 3.

FIG. 6 is a SEM photograph of the surface of the artificial blood vessel.

### EMBODIMENT FOR CARRYING OUT THE INVENTION

The artificial blood vessel according to one embodiment of the present invention will be described below with reference to the drawings. Besides, embodiments shown below are merely examples, and the artificial blood vessel of the present invention is not limited to the following embodiments.

It should be noted that, in the present specification, “perpendicular to A” and similar expressions do not only refer to a direction strictly perpendicular to A, but also refer to the direction including being substantially perpendicular to A. Moreover, in the present specification, “parallel to B” and similar expressions do not only refer to a direction strictly parallel to B, but also refer to the direction including being substantially parallel to B. In addition, in the present specification, “C-shape” and similar expressions do not only refer to a strict C-shape, but also refer to the shape including a shape visually associated with a C-shape (substantially a C-shape).

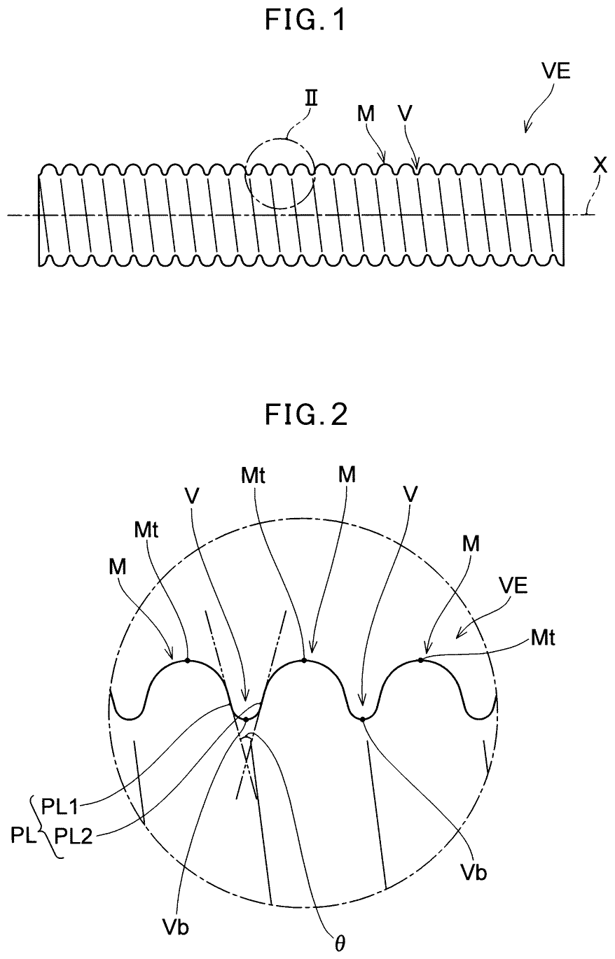

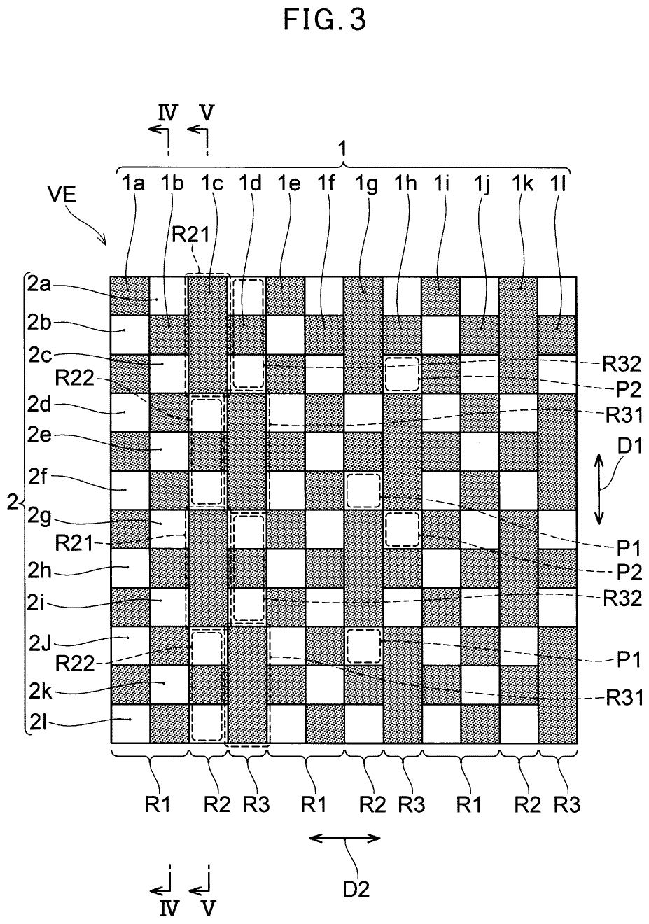

FIG. 1 is a side view of an artificial blood vessel according to one embodiment of the present invention. FIG. 2 is a partially enlarged view of a region II of the artificial blood vessel in FIG. 1. FIG. 3 is a fabric structure diagram showing one example of a weaving structure of a base material used in the artificial blood vessel of FIG. 1.

The artificial blood vessel is used, such as, for example, for replacing a pathological living blood vessel and bypassing the living blood vessel. As shown in FIGS. 1 and 2, in an artificial blood vessel VE of the present embodiment, mountain parts M and valley parts V are alternately formed in an axial direction X (see FIG. 1) of the artificial blood vessel VE. By the configuration in which the mountain parts M and the valley parts V are alternately formed in the artificial blood vessel VE, the artificial blood vessel becomes flexible and is not easy to kink when the artificial blood vessel VE is bent. In the present embodiment, although the artificial blood vessel VE is formed in a cylindrical shape with the mountain parts M and the valley parts V formed in a spiral shape, the artificial blood vessel may not have the mountain parts M and valley parts V.

A diameter of the artificial blood vessel VE can be changed depending on a site to be used, etc., and is not particularly limited. For example, the artificial blood vessel VE may be an artificial blood vessel with a large diameter having an inner diameter of 10 mm or more (for a thoracoabdominal aorta), an artificial blood vessel with a medium diameter having an inner diameter of 6 mm or more and less than 10 mm, such as 6 mm and 8 mm (for arteries in lower limb, neck, and axillary regions), or an artificial blood vessel with a small diameter having an inner diameter of less than 6 mm. A thickness of the artificial blood vessel VE is appropriately changed depending on an inner diameter and a length of the artificial blood vessel to be used and is not particularly limited. For example, the thickness of the artificial blood vessel VE can be 0.1 to 2 mm.

A length of the artificial blood vessel VE in the axial direction X can be changed depending on a site to be used and is not particularly limited. For example, the length of the artificial blood vessel VE in the axial direction X can be 100 to 1000 mm. It should be noted that, when the artificial blood vessel VE is implanted to a desired site, the artificial blood vessel VE is cut to a predetermined length and used by a physician, etc. Depending on a site to be implanted, the artificial blood vessel VE may be cut perpendicular to the axial direction X or may be cut inclining diagonally at a predetermined angle to the axial direction X.

When the artificial blood vessel VE has mountain parts M and valley parts V, the number of mountain parts M (or valley parts V) (the number of pleats) of the artificial blood vessel VE is not particularly limited, and may be appropriately set depending on a required kink resistance performance. For example, the number of mountain parts M (the number of pleats) of the artificial blood vessel VE can be 20 to 70, preferably 25 to 35, per 100 mm length in the axial direction X in a case of an artificial blood vessel having an outer diameter of 15 mm. Moreover, an interval (pitch) in the axial direction X between an top part Mt (see FIG. 2) of a mountain part M and an top part Mt of an adjacent mountain part M of the artificial blood vessel VE is not particularly limited, and the interval can be, for example, 10 to 30%, preferably 15 to 25%, of an outer diameter (an outer diameter at the top part Mt of the mountain part M) of the artificial blood vessel VE. Furthermore, a depth from the top part Mt of the mountain part M to a bottom part Vb (see FIG. 2) of the valley part V is not particularly limited, and the depth can be, for example, 5 to 20%, preferably 5 to 15%, of the outer diameter of the artificial blood vessel VE.

In addition, in the present embodiment, a curvature at the top part Mt of the mountain part M is smaller than a curvature at the bottom part Vb of the valley part V (in the present embodiment, a radius of curvature at the top part Mt of the mountain part M is larger than a radius of curvature at the bottom part Vb of the valley part V). It should be noted that the phrase “the curvature at the top part Mt of the mountain part M is smaller than the curvature at the bottom part Vb of the valley part V” means that a degree of curve along the axial direction X at the top part Mt of the mountain part M is smaller than a degree of curve along the axial direction X at the bottom part Vb of the valley part V (the curve of the mountain part M is more gradual than the curve of the valley part V), and the mountain part M and the valley part V do not need to form perfect arc surfaces. When the curvature at the top part Mt of the mountain part M is smaller than the curvature at the bottom part Vb of the valley part V, stress is concentrated at the valley part V when an external force is applied to the artificial blood vessel VE. Therefore, the artificial blood vessel VE becomes easy to curve starting from the valley part V. The curvatures of the mountain part M and valley part V are not particularly limited. For example, the radius of curvature of the top part Mt of the mountain part M can be 5 to 8% of the diameter of the artificial blood vessel VE (and can be larger than the radius of curvature of the bottom part Vb of the valley part V). Moreover, the radius of curvature of the bottom part Vb of the valley part V can be 2 to 3% of the diameter of the artificial blood vessel VE (and can be smaller than the radius of curvature of the top part Mt of the mountain part M). With the artificial blood vessel VE being easy to curve, the curved artificial blood vessel VE is less likely to return to its original state, so that a load on a connection site between the artificial blood vessel VE and a blood vessel, etc. can be reduced.

A curved part at the top part Mt of the mountain part M and a curved part at the bottom part Vb of the valley part V can be connected by a planar part PL (see FIG. 2). Flexibility and kink resistance can be thereby further improved compared to a case where the curved parts are directly connected to each other. An angle θ between a planar part PL1 on one side and a planar part PL2 on the other side can be 20° to 40°, preferably 30°, and the angle θ between the planar part PL1 on one side and the planar part PL2 on the other side can be appropriately set depending on a diameter of the artificial blood vessel, a height of the mountain part, a height of the valley part, a pitch, etc.

Next, a configuration of the base material constituting the artificial blood vessel VE will be described.

The artificial blood vessel VE of the present embodiment has a predetermined weaving structure in which a warp yarn 1 and a weft yarn 2 are woven. The predetermined weaving structure of the artificial blood vessel VE can adopt a publicly-known weaving structure that can be used for an artificial blood vessel or a combination of publicly-known weaving structures. For example, the artificial blood vessel VE may have, in whole or in part, a plain weave structure, a twill weave structure, a satin weave structure, or a combination of these weave structures. In the present embodiment, at least one of the warp yarn 1 and the weft yarn 2 that constitute the artificial blood vessel VE is composed of a multifilament yarn including a plurality of filament yarns. It should be noted that only one of the warp yarn 1 and the weft yarn 2 may be composed of a multifilament yarn, or both of the warp yarn 1 and the weft yarn 2 may be composed of a multifilament yarn.

In the present embodiment, as shown in FIG. 3, the artificial blood vessel VE has warp yarns 1a to 1l (hereinafter collectively referred to as warp yarn 1) extending along the axial direction X (up down direction in FIG. 3) and weft yarns 2a to 2l (hereinafter collectively referred to as weft yarn 2) extending along a circumferential direction (left right direction in FIG. 3) of the artificial blood vessel VE. More specifically, as shown in FIG. 3, the artificial blood vessel VE has a plurality of warp yarns 1a to 1l and a plurality of weft yarns 2a to 2l, and has a weaving structure in which the warp yarn 1 and the weft yarn 2 are interlaced. In FIG. 3, the warp yarn 1 extends in the up down direction, and the extending direction of the warp yarn 1 (axial direction X of the artificial blood vessel VE) is referred to as D1. Moreover, in FIG. 3, the weft yarn 2 extends in the left right direction, and the extending direction of the weft yarn 2 (circumferential direction of the artificial blood vessel VE) is referred to as D2. In FIG. 3, shown in black (dotted parts) are parts where the warp yarn 1 is exposed on the outer surface (front surface) of the artificial blood vessel VE, and shown in white are parts where the weft yarn 2 is exposed on the outer surface of the artificial blood vessel VE. A loom for producing the artificial blood vessel E is not particularly limited.

As will be mentioned later, in the present embodiment, the warp yarn 1 has portions R21 and R31 that extend so as to cross over a plurality of weft yarn 2 (see FIGS. 3 and 5). Specifically, as shown in FIG. 3, the warp yarn 1 has portions R21 and R31 that extend so as to cross over a plurality of weft yarns 2 and portions R1, R22, and R32 that extend so as to cross over a single weft yarn 2. It should be noted that the warp yarn 1 do not necessarily have a portion that extends so as to cross over a plurality of weft yarns 2. Moreover, when the warp yarn 1 has a portion that extends so as to cross over a plurality of weft yarns 2, the weaving structure of the artificial blood vessel VE is not necessarily limited to the weaving structure shown in FIG. 3, and may have another weaving structure.

In the present embodiment, the artificial blood vessel VE has a first region R1 in which the warp yarn 1 and the weft yarn 2 are woven in a plain weave, as shown in FIG. 3. Moreover, the artificial blood vessel VE has a second region R2 having a first portion on a second region side R21 where the warp yarn 1 crosses over a plurality of weft yarns 2 (portion that extends so as to cross over a plurality of weft yarns 2), and a second portion on the second region side R22 where the warp yarn 1 extends so as to cross over a single weft yarn 2 (portion that extends so as to cross over a single weft yarn 2) on one surface of the artificial blood vessel VE (outer surface (front surface) of the artificial blood vessel VE, in the present embodiment). Moreover, the artificial blood vessel VE has a third region R3 having a first portion on a third region side R31 where the warp yarn 1 crosses over a plurality of weft yarns 2 (portion that extends so as to cross over a plurality of weft yarns 2), and a second portion on the third region side R32 where the warp yarn 1 extends so as to cross over a single weft yarn 2 (portion that extends so as to cross over a single weft yarn 2) on one surface of the artificial blood vessel VE (outer surface of the artificial blood vessel VE, in the present embodiment). The first region R1, the second region R2, and the third region R3 are alternately formed in the extending direction D2 of the weft yarn 2, as shown in FIG. 3. That is, the first region R1, the second region R2, and the third region R3 are repeatedly arranged in this order in the extending direction D2 of the weft yarn 2. The first portion on the second region side R21 is adjacent to the second portion on the third region side R32 in the extending direction D2 of the weft yarn 2, and the second portion on the second region side R22 is adjacent to the first portion on the third region side R31 in the extending direction D2 of the weft yarn 2. In the present embodiment, the warp yarn 1 is composed of a multifilament yarn. When the artificial blood vessel VE of the present embodiment has the above-described configuration, the warp yarn 1 composed of a multifilament yarn, which extends long without being restrained in the first portion on the second region side R21 or the first portion on the third region side R31, spreads toward the first region R1 woven in a plain weave, as will be mentioned later. With this three-dimensional structure of the warp yarn 1, when blood seeps out from a gap between fibers generated in the first region R1 woven in a plain weave, the blood is suppressed from leaking out and retained in the three-dimensional structure. By coagulating the blood with being retained, blood leakage resistance can be improved. A configuration and a weaving structure of each part of the artificial blood vessel VE will be described below.

The warp yarn 1 is a fiber extending in one direction, among fibers constituting the artificial blood vessel VE. In the present embodiment, the warp yarn 1 is a fiber extending along a length direction (axial direction X) of the artificial blood vessel VE. The warp yarn 1 is made of a material applicable to a fabric artificial blood vessel composed of a weaving structure of fibers. The material of the warp yarn 1 is not particularly limited as long as it is a material applicable to the fabric artificial blood vessel. For example, the material of the warp yarn 1 can be polyester, polytetrafluoroethylene, polyamide, or the like. Moreover, as the material of the warp yarn 1, a composite material composed of two or more kinds of applicable materials having different properties such as a melting point and a degree of shrinkage may be used. For example, the material of the warp yarn 1 can be a synthetic fiber in which polyethylene terephthalate (PET) and polytrimethylene terephthalate (PTT), etc. are combined at a spinning stage to form one long filament having a spiral crimp. For example, when the composite material composed of two kinds of materials having properties of different melting point and degree of shrinkage, which has a spiral crimp, is used as the material of the warp yarn 1, a three-dimensional structure composed of the warp yarn 1 which will be mentioned later is easy to spread in the extending direction D2 of the weft yarn 2, further enhancing a performance of retaining blood, which can improve blood leakage resistance.

Each warp yarn 1 may be a monofilament yarn or a multifilament yarn, but in the present embodiment, the warp yarn 1 is composed of multifilament yarn. When the warp yarn 1 is a monofilament yarn, the weft yarn 2 is composed of a multifilament yarn. A fineness of the warp yarn 1 is not particularly limited, but for example, when the warp yarn 1 is a monofilament yarn, a single yarn fineness of the warp yarn can be 15 to 100 dtex, preferably 20 to 75 dtex. Moreover, when the warp yarn 1 is a multifilament yarn, a fineness of the warp yarn 1 can be, for example, 0.25 to 2.50 dtex, preferably 0.50 to 2.00 dtex, for a single yarn fineness of the warp yarn 1, and can be 2 to 2500 dtex, preferably 6 to 1600 dtex, more preferably 10 to 540 dtex, and further preferably 30 to 200 dtex, for a total fineness of warp yarn 1. When the single yarn fineness of the warp yarn 1 and the total fineness of the warp yarn 1 are within the above-described ranges, the warp yarn 1 in the second region R2 and the third region R3 can be satisfactorily spread toward the first region R1. Thus, when blood seeps out from a gap in the first region R1 by the warp yarn 1 in the second region R2 and the third region R3, the blood is suppressed from leaking out, retained by the three-dimensional structure of the warp yarn 1, and coagulates in the retained state, which can improve blood leakage resistance. It should be noted that a “single yarn fineness” is a fineness per single filament constituting the warp yarn 1, and a “total fineness” is a product of the single yarn fineness and the number of filaments constituting the warp yarn 1. The number of filament yarns (hereinafter referred to as the number of filaments) constituting one warp yarn is not particularly limited. For example, as will be mentioned later, when the total number of filaments of the warp yarn 1 is 1.5 times or more the number of filaments per single weft yarn 2 and the number of the warp yarn 1 crossing over a plurality of weft yarns 2 is one in the second region R2, the number of filaments per single warp yarn 1 can be 8 to 1000, preferably 12 to 800, more preferably 20 to 270, and further preferably 60 to 100. As will be mentioned later, when the number of filaments per single warp yarn 1 is 0.8 to 1.2 times the number of filaments per single weft yarn 2 and the number of the warp yarns 1 crossing over a plurality of weft yarns 2 is two or more in the second region R2, the number of filaments per single yarn 1 can be 4 to 500, preferably 6 to 400, more preferably 10 to 135, and further preferably 30 to 50.

The weft yarn 2 is a fiber extending in a direction intersecting with the warp yarn 1, among fibers constituting the artificial blood vessel VE. In the present embodiment, the weft yarn 2 is a fiber extending in a circumferential direction of the artificial blood vessel VE. The weft yarn 2 is made of a material applicable to a fabric artificial blood vessel composed of a weaving structure of fibers. The material of the weft yarn 2 is not particularly limited as long as it is a material applicable to the fabric artificial blood vessel. For example, the material of the weft yarn 2 can be polyester, polytetrafluoroethylene, polyamide, or the like.

Each weft yarn 2 may be a monofilament yarn or a multifilament yarn, but in the present embodiment, the weft yarn 2 is composed of a multifilament yarn. When the weft yarn 2 is a monofilament yarn, the warp yarn 1 is composed of a multifilament yarn. A fineness of the weft yarn 2 is not particularly limited, but for example, when the weft yarn 2 is a monofilament yarn, a single yarn fineness of the weft yarn can be 15 to 100 dtex, preferably 20 to 75 dtex. Moreover, when each weft yarn 2 is composed of a multifilament yarn, for example, the single yarn fineness of the weft yarn 2 can be 0.25 to 2.50 dtex, preferably 0.50 to 2.00 dtex, and a total fineness of the weft yarn 2 can be 1 to 1250 dtex, preferably 3 to 800 dtex, more preferably 5 to 270 dtex, and further preferably 15 to 100 dtex. It should be noted that the “single yarn fineness” is a fineness per single filament (monofilament or multifilament) constituting the weft yarn 2, and the “total fineness” is a product of the single yarn fineness and the number of filaments constituting weft yarn 2. When the weft yarn 2 is composed of a multifilament yarn, the number of filament yarns constituting one weft yarn can be 4 to 500, preferably 6 to 400, more preferably 10 to 135, and further preferably 30 to 50.

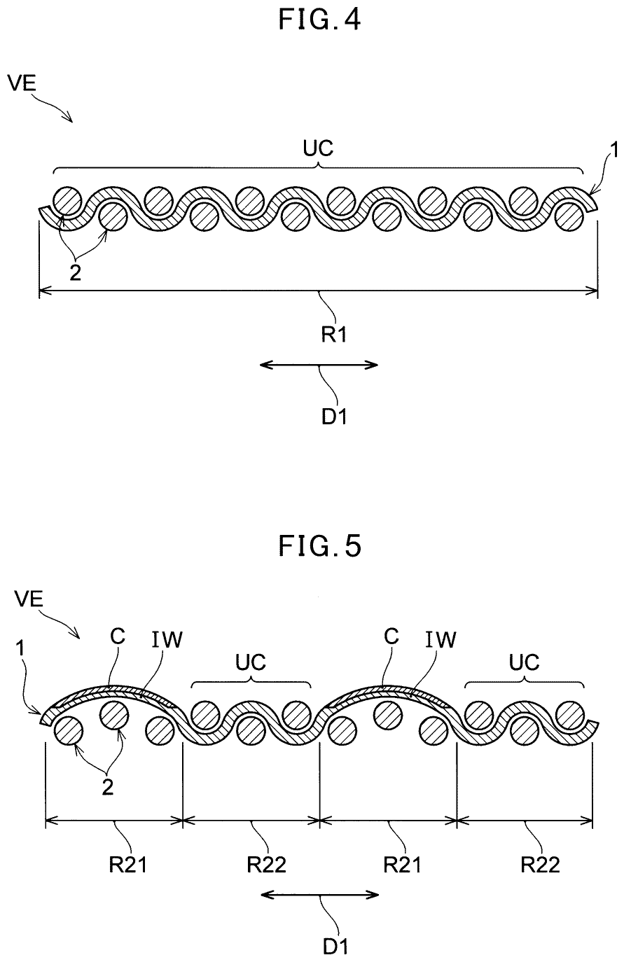

The first region R1 is a part where the warp yarn 1 and the weft yarn 2 are plain-woven. In FIG. 3, the first region R1 is a region where the warp yarns 1a, 1b, 1e, 1f, 1i, 1j and the weft yarn 2 (weft yarns 2a to 2l) intersect. In the first region R1 of the plain weave structure, the warp yarn 1 extends so as to cross over only one weft yarn 2 (not to cross over a plurality of weft yarns 2) moving from one surface of the artificial blood vessel VE (the outer surface (front surface) of the artificial blood vessel VE, which is the upper surface in FIG. 4) to the other surface of the artificial blood vessel VE (the inner surface of the artificial blood vessel VE, which is the lower surface in FIG. 4) and from the other surface to one surface, as shown in FIG. 4. The first region R1 improves a strength of the artificial blood vessel VE, particularly a tensile strength (in the axial direction X of the artificial blood vessel VE). The first region R1 extends along the extending direction D1 of the warp yarn 1 and extends in the axial direction X of the artificial blood vessel VE. Moreover, a plurality of first regions R1 are arranged apart from each other at a predetermined interval in the extending direction D2 of the weft yarn 2. The second region R2 and the third region R3 are arranged between one first region R1 and the other first region R1 in the extending direction D2 of the weft yarn 2.

In the present embodiment, as shown in FIG. 3, two warp yarns 1a, 1b (warp yarns 1e, 1f, or warp yarns 1i, 1j) and a plurality of weft yarns 2a to 2l (and weft yarns not shown) are plain-woven in the first region R1. The number of warp yarns 1 provided in one first region R1 can be 2 to 4, preferably 2 to 3, and more preferably 2. It should be noted that in the present specification, when the warp yarn 1 is a multifilament yarn, the term “the number of warp yarns” refers to, with the warp yarn 1 composed of a plurality of filament yarns to be bundled being as one, the number of warp yarns 1 in which the filament yarns are bundled, not the number of filaments constituting multifilament yarns. When the number of warp yarns 1 is within the above-mentioned ranges, an uncovered area of the first region R1, which is not covered by the warp yarn 1 in the first portion on the second region side R21 and the warp yarn 1 in the first portion on the third region side R31, can be reduced. Therefore, the first region R1 in plain weave becomes easily covered three-dimensionally with the warp yarn 1 in the first portion on the second region side R21 and the warp yarn 1 in the first portion on the third region side R31. When blood seeps out from the first region R1, the blood is retained by the three-dimensional structure of the warp yarn 1 in the first portion on the second region side R21 and warp yarn 1 in the first portion on the third region side R31 and coagulates in the retained state, so that an amount of blood leakage from the artificial blood vessel VE can be reduced. Moreover, in the artificial blood vessel VE, a ratio of the number of warp yarns 1 in the first region R1 to the total number of warp yarns 1 arranged in the extending direction D2 of the weft yarn 2 in the first region R1 to the third region R3 (the number of warp yarns in the first region R1/the total number of warp yarns) is not particularly limited, but can be, for example, 0.2 to 0.4 (⅓ in the present embodiment). When the number of warp yarns 1 in the first region R1 and the ratio of the number of warp yarns 1 are within the above-described ranges, the amount of blood leakage from the artificial blood vessel VE can be reduced while increasing the strength of the artificial blood vessel VE.

The second region R2 has a first portion on a second region side R21 in which the warp yarn 1 crosses over a plurality of weft yarns 2 and a second portion on a second region side R22 in which the warp yarn 1 extends so as to cross over one weft yarn 2. As shown in FIG. 3, the first portion on the second region side R21 and the second portion on the second region side R22 are alternately provided in the extending direction D1 of the warp yarn 1. Since the second region R2 has the first portion on the second region side R21 and the second portion on the second region side R22, the artificial blood vessel VE can be made more flexible as compared with artificial blood vessel VE, all region of which has a plain weave structure. It should be noted that a portion of a warp yarn 1c provided in the second region R2 may be composed of one warp yarn or a plurality of warp yarns. The number of warp yarns 1 provided in the second region R2 can be, for example, 1 to 4, preferably 2 to 3, and more preferably 2.

The first portion on the second region side R21 is a portion woven so that the warp yarn 1 has a portion crossing over a plurality of weft yarns 2. In the present embodiment, the warp yarns 1c, 1g, 1k, etc. cross over the plurality of weft yarns 2. In the first portion on the second region side R21, the warp yarn 1 crosses over the plurality of weft yarns 2, so that the artificial blood vessel VE becomes more flexible in that portion than in the plain weave structure. Moreover, when the warp yarn 1 in the first portion on the second region side R21 is composed of a multifilament yarn, both ends of the first portion on the second region side R21 in the extending direction D1 of the warp yarn 1 become in a state of being bound by the weft yarns 2 in the second portion on the second region side R22 (see the part PI in FIG. 3). In this case, the first portion on the second region side R21 of the warp yarn 1 composed of a multifilament yarn, both ends of which are bound, forms a three-dimensional structure in which a central part in the extending direction D1 of the warp yarn 1 spreads in the extending direction D2 of the weft yarn 2 (note that this three-dimensional structure also spreads in the left right direction and in a front direction of a paper surface in FIG. 3). Thus, the first region R1 with the plain weave structure adjacent to the first portion on the second region side R21 in the extending direction D2 of the weft yarn 2 is partially covered by the spread multifilament yarns in the first portion on the second region side R21. With this three-dimensional structure of the warp yarn 1, when blood seeps out from a gap between fibers generated in the plain-woven first region R1, the seeping blood is retained in a gap between filaments of the three-dimensional structure composed of the multifilament. The blood thereby coagulates in the retained state, so that blood leakage resistance can be improved. Moreover, in the present embodiment, the second portion on the third region side R32 adjacent to the first portion on the second region side R21 in the extending direction D2 of the weft yarn 2 is also partially covered by the spread multifilament yarns in the first portion on the second region side R21. As a result, a gap generated in the second portion on the third region side R32 is also covered by the multifilament yarn in the first portion on the second region side R21, so that the blood in the artificial blood vessel VE becomes less likely to leak out to the outside.

In the first portion on the second region side R21 (from a portion where the warp yarn 1 exits from the other surface of the artificial blood vessel VE to one surface of the artificial blood vessel VE (the surface shown in FIG. 3), to a portion where the warp yarn 1 enters into the other surface), the number of weft yarns 2 that the warp yarn 1 crosses over is not particularly limited, but can be, for example, 2 to 5, preferably 3 to 4, more preferably 3 (the state shown in FIG. 3). When the number of warp yarns 2 that the warp yarn 1 crosses over is within the above-described ranges, in the first portion on the second region side R21, the multifilament yarn of the warp yarn 1 is easy to be spread in the extending direction D2 of the weft yarn 2, and the strength of the artificial blood vessel VE can be maintained at a predetermined level.

In the first portion on the second region side R21, the number of warp yarns 1 constituting the first portion on the second region side R21 is not particularly limited as long as the warp yarn 1 has a portion crossing over a plurality of weft yarns 2. For example, the first portion on the second region side R21 (the second region R2) may be composed of a plurality of (two) warp yarns (each of warp yarns 1c, 1g, and 1k is composed of a plurality of warp yarns). Moreover, the first portion on the second region side R21 (the second region R2) may have at least one warp yarn 1 extending so as to cross over (only) one weft yarn 2 and at least one warp yarn 1 crossing over a plurality of weft yarns 2.

The second portion on the second region side R22 is a portion woven so that the warp yarn 1 crosses over only one weft yarn 2 (the warp yarn 1 does not cross over a plurality of weft yarns 2 from a portion where it exits from the other surface of the artificial blood vessel VE to one surface of the artificial blood vessel VE (the surface shown in FIG. 3) to a portion where it enters into the other surface). The second portion on the second region side R22 is set to have the same degree of length as the length of the first portion on the second region side R21 in the extending direction D1 of the warp yarn 1. That is, the number of weft yarns 2 in the first portion on the second region side R21 (three weft yarns 2 in FIG. 3) is equal to the number of weft yarns 2 in the second portion on the second region side R22 (three weft yarns 2 in FIG. 3).

The third region R3 has a first portion on a third region side R31 in which the warp yarn 1 crosses over a plurality of weft yarns 2 and a second portion on a third region side R32 in the which warp yarn 1 extends so as to cross over one weft yarn 2. As shown in FIG. 3, the first portion on the third region side R31 and the second portion on the third region side R32 are alternately provided in the extending direction D1 of the warp yarn 1. Since the third region R3 has the first portion on the third region side R31 and the second portion on the third region side R32, the artificial blood vessel VE can be made more flexible as compared with the artificial blood vessel VE, all region of which has a plain weave structure. It should be noted that a portion of a warp yarn 1d provided in the third region R3 may be composed of single warp yarn or a plurality of warp yarns. The number of warp yarns 1 provided in the third region R3 can be, for example, 1 to 4, preferably 2 to 3, and more preferably 2.

The first portion on the third region side R31 is a portion woven so that the warp yarn 1 has a portion crossing over a plurality of weft yarns 2. In the present embodiment, the warp yarns 1d, 1h, 1l, etc. cross over the plurality of weft yarns 2. In the first portion on the third region side R31, the warp yarn 1 crosses over the plurality of weft yarns 2, so that the artificial blood vessel VE becomes more flexible in that part than in the plain weave structure. Moreover, when the warp yarn 1 of the first portion on the third region side R31 is composed of a multifilament yarn, both ends of the first portion on the third region side R31 in the extending direction D1 of the warp yarn 1 become in a state of being bound by the weft yarn 2 in the second portion on the third region side R32 (see the part P2 in FIG. 3). In this case, the first portion on the third region side R31 of the warp yarn 1 composed of a multifilament yarn, both ends of which are bound, forms a three-dimensional structure in which a central part in the extending direction D1 of the warp yarn 1 spreads in the extending direction D2 of the weft yarn 2. Thus, the first region R1 with the plain weave structure adjacent to the first portion on the third region side R31 in the extending direction D2 of the weft yarn 2 is partially covered by the spread multifilament yarn in the first portion on the third region side R31. With this three-dimensional structure of the warp yarn 1, when blood seeps out from a gap between fibers generated in the plain-woven first region R1, the seeping blood is retained in a gap between filaments of the three-dimensional structure composed of multifilament. The blood thereby coagulates in the retained state, so that blood leakage resistance can be improved. Moreover, in the present embodiment, the second portion on the second region side R22 adjacent to the first portion on the third region side R31 in the extending direction D2 of the weft yarn 2 is also partially covered by the spread multifilament yarn in the first portion on the third region side R31. As a result, a gap generated in the second portion on the second region side R22 is also covered by the multifilament yarn in the first portion on the third region side R31, so that the blood in the artificial blood vessel VE becomes less likely to leak out to the outside.

In the first portion on the third region side R31 (from a portion where the warp yarn 1 exits from the other surface of the artificial blood vessel VE to one surface of the artificial blood vessel VE (the surface shown in FIG. 3), to a portion where the warp yarn 1 enters into the other surface), the number of weft yarns 2 that the warp yarn 1 crosses over is not particularly limited, but can be, for example, 2 to 5, preferably 3 to 4, more preferably 3 (the state shown in FIG. 3). When the number of weft yarns 2 that the warp yarn 1 crosses over is within the above-described ranges, in the first portion on the third region side R31, the multifilament yarn of the warp yarn 1 is easy to be spread in the extending direction D2 of the weft yarn 2, and the strength of the artificial blood vessel VE can be maintained at a predetermined level.

In the first portion on the third region side R31, the number of warp yarns 1 constituting the first portion on the third region side R31 is not particularly limited as long as the warp yarn 1 has a portion crossing over a plurality of weft yarns 2. For example, the first portion on the third region side R31 (the third region R3) may be composed of a plurality of (two) warp yarns (each of warp yarns 1d, 1h, and 1l is composed of a plurality of warp yarns). Moreover, the first portion on the third region side R31 (the third region R3) may have at least one warp yarn 1 extending so as to cross over (only) one weft yarn 2 and at least one warp yarn 1 crossing over a plurality of weft yarns 2.

The second portion on the third region side R32 is a portion woven so that the warp yarn 1 crosses over only one weft yarn 2 (the warp yarn 1 does not cross over a plurality of weft yarns 2 from a portion where it exits from the other surface of the artificial blood vessel VE to one surface of the artificial blood vessel VE (the surface shown in FIG. 3) to a portion where it enters into the other surface). The second portion on the third region side R32 is set to have the same degree of length as the length of the first portion on the third region side R31 in the extending direction D1 of the warp yarn 1. That is, the number of weft yarns 2 in the first portion on the third region side R31 (three weft yarns 2 in FIG. 3) is the same as the number of weft yarns 2 in the second portion on the third region side R32 (three weft yarns 2 in FIG. 3).

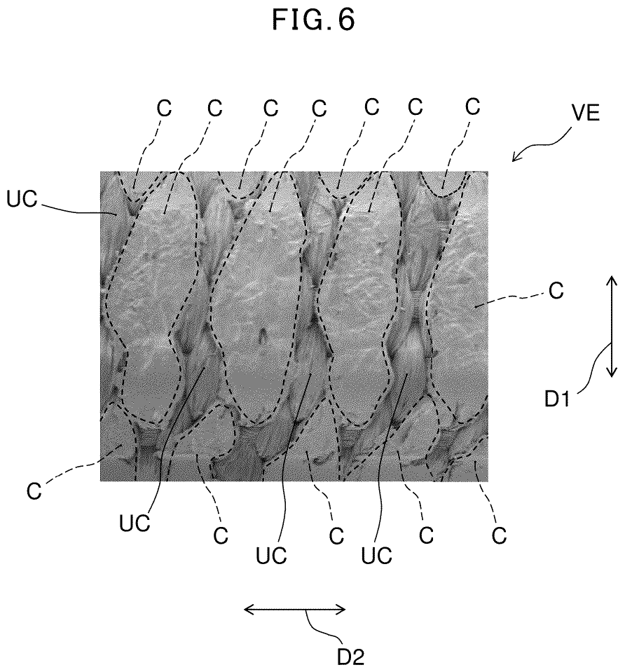

As shown in FIGS. 5 and 6, the artificial blood vessel VE of the present embodiment has a plurality of covering parts C provided at a plurality of locations on a surface of the artificial blood vessel VE, each of the plurality of covering parts C covering multifilament yarn in a planar shape, and uncovered parts UC that are provided between the plurality of covering parts C and are not covered by the covering parts C on the surface of the artificial blood vessel VE.

The covering parts C partially cover a plurality of filament yarns of the multifilament yarn constituting one warp yarn or one weft yarn on the surface of the artificial blood vessel VE. “Covering multifilament yarn in a planar shape” means that the covering part C extends in an extending direction of the multifilament yarn (extending direction D1 of the warp yarn 1) and in a direction perpendicular to the extending direction of the multifilament yarn (extending direction D2 of the weft yarn 2) on the surface of the artificial blood vessel VE so as to fill gaps on the surface side of the artificial blood vessel VE between a plurality of adjacent filament yarns that constitute the multifilament yarn. By providing the covering parts C, the gaps between the plurality of filament yarns located inside the covering parts C in the radial direction of the artificial blood vessel VE are filled on the surface of the artificial blood vessel VE, details of which will be mentioned later. This improves blood leakage resistance of the artificial blood vessel VE.

As shown in FIGS. 5 and 6, the covering parts C are provided at a plurality of locations on the surface of the artificial blood vessel VE. Here, “a plurality of locations” means that, when the entire surface of the artificial blood vessel VE is divided into a plurality of parts, the covering parts C are provided on the plurality of parts. The covering parts C may be provided at a plurality of locations with being separated from each other, or may be provided at a plurality of locations continuously with each other in an axial direction (extending direction D1 of the warp yarn 1) and/or a circumferential direction (extending direction D2 of the weft yarn 2) of the artificial blood vessel VE.

As shown in FIG. 6, the uncovered parts UC are remaining parts with respect to the covering parts C on the surface of the artificial blood vessel VE that are not covered by the covering parts C. As shown in FIG. 6, the uncovered parts UC are arranged between the covering parts C provided at a plurality of locations. The uncovered parts UC are arranged between the covering parts C, for example, in the axial direction (extending direction D1 of the warp yarn 1) and/or the circumferential direction (extending direction D2 of the weft yarn 2) of the artificial blood vessel VE. The uncovered parts UC are arranged between the covering parts C on the surface of the artificial blood vessel VE, thereby contributing to maintaining flexibility of the artificial blood vessel VE, details of which will be mentioned later. In the present embodiment, multifilament yarn of the warp yarn 1 and the weft yarn 2 in regions of the uncovered parts UC is exposed on the surface of the artificial blood vessel VE with gaps maintained between adjacent filament yarns (see FIG. 6).

As mentioned above, the artificial blood vessel VE of the present embodiment has a plurality of covering parts C provided at a plurality of locations on the surface of the artificial blood vessel VE, each of the plurality of covering parts C covering a plurality of multifilament yarns in a planar shape, and uncovered parts UC that are provided between the plurality of covering parts C and are not covered by the covering parts C on the surface of the artificial blood vessel VE. Therefore, the gaps between the plurality of filament yarns that constitute the multifilament yarn are filled by the covering parts C, so that blood leakage resistance of the artificial blood vessel VE is improved. Moreover, by providing the uncovered parts UC between the covering parts C at a plurality of locations, flexibility of the artificial blood vessel VE as a whole can be maintained. Thus, according to the artificial blood vessel VE of the present embodiment, blood leakage resistance can be improved while maintaining flexibility of the artificial blood vessel VE.

A ratio of the covering parts C to the surface area of the artificial blood vessel VE is not particularly limited, but it is preferable that a total area of a plurality of covering parts C is 50% to 90%, more preferably 60 to 80%, of the surface area of the artificial blood vessel VE. In this case, flexibility of the artificial blood vessel VE can be maintained while further improving blood leakage resistance of the artificial blood vessel VE.

Moreover, in the present embodiment, as shown in FIG. 5, the artificial blood vessel VE has inner weaving parts IW in which a plurality of filament yarns of multifilament yarn extend with being separated from each other on the inner side in the radial direction of the artificial blood vessel VE with respect to the covering parts C (lower side in FIG. 5). The inner weaving part IW constitutes a part of a weaving structure of the artificial blood vessel VE and is covered by the covering part C with a plurality of filament yarns separated from each other with gaps therebetween. The plurality of filament yarns of the inner weaving part IW are schematically shown in FIG. 5, but they extend in a bundle such that they are arranged adjacent in the radial direction of the artificial blood vessel VE (up down direction in FIG. 5) and arranged adjacent in the extending direction D2 of the weft yarn 2 (depth direction of the paper in FIG. 5). The inner weaving part IW is covered by the covering part C and cannot be seen in FIG. 6, but it is located in the depth direction of the paper with respect to the covering part C. A structure of the inner weaving part IW is not particularly limited as long as a plurality of filament yarns extend on the inner side in the radial direction of the artificial blood vessel VE with respect to the covering part C with the filament yarns being separated from each other. In the present embodiment, the inner weaving part IW has a structure in which multifilament yarn in a region corresponding to the first portion on the second region side R21 (and the first portion on the third region side R31) spreads in the extending direction D2 of the weft yarn 2, and the plurality of filament yarns that constitute the multifilament yarn of the inner weaving part IW extend along the extending direction D1 of the warp yarn 1 with the filament yarns being separated from each other. In the present embodiment, as shown in FIG. 5, the warp yarn 1 has a two-layer structure of covering part C that is a planar resin layer on the surface side of the artificial blood vessel VE and inner weaving part IW that is multifilament layer located on the inner side in the radial direction with respect to covering layer C, in the region corresponding to the first portion on the second region side R21 (and the first portion on the third region side R31).

With the inner weaving part IW being provided on the inner side in the radial direction of the covering part C, the plurality of filament yarns extend with being separated from each other so as to have gaps therebetween, on the inner side in the radial direction of the planar covering part C. Thus, the inner weaving parts IW, composed of multifilament yarn covered by the covering parts C, extend while maintaining a predetermined flexibility. Thus, even if the planar covering part C is provided, flexibility of the artificial blood vessel VE as a whole is less likely to be impaired, and it is possible to achieve both flexibility and blood leakage resistance of the artificial blood vessel VE. Moreover, by having the inner weaving parts IW, the inner structure of the artificial blood vessel can maintain its weaving structure and can be suppressed from being impaired in cell invasiveness.

Moreover, the covering parts C and the uncovered parts UC are preferably provided alternately in the axial direction (extending direction D1 of the warp yarn 1) and/or the circumferential direction (extending direction D2 of the weft yarn 2) of the artificial blood vessel VE, on a part of the surface of the artificial blood vessel VE. In this case, the covering parts C and the uncovered parts UC are arranged with a good balance in the axial direction and/or the circumferential direction of the artificial blood vessel VE. Thus, flexibility and blood leakage resistance of the artificial blood vessel VE are improved with a good balance, and the artificial blood vessel VE is suppressed from becoming locally hardened and becoming locally susceptible to blood leakage. In particular, when the covering parts C and the uncovered parts UC are provided alternately in the axial direction of the artificial blood vessel VE, the artificial blood vessel VE becomes easy to be bent, facilitating arrangement of the artificial blood vessel VE within the body. Moreover, when the covering parts C and the uncovered parts UC are provided alternately in the circumferential direction of the artificial blood vessel VE, the artificial blood vessel VE becomes easy to be twisted, suppressing deformation (collapse) of the artificial blood vessel VE when twisted. Thus, even if the artificial blood vessel VE is subjected to a force in a twisting direction due to screwing, for example, when connected to an artificial heart-lung machine or the like, it is suppressed from being collapsed due to twisting. Thus, adverse effects caused by twisting of the artificial blood vessel VE, such that a blood coagulates at a collapsed part to occlude the artificial blood vessel VE due to twisting and collapsing of the artificial blood vessel VE, are suppressed. In the present embodiment, the artificial blood vessel VE has covering parts C and uncovered parts UC alternately provided in both the axial direction and the circumferential directions of the artificial blood vessel VE. In this case, flexibility and blood leakage resistance of the artificial blood vessel VE are improved with a good balance throughout the entire artificial blood vessel VE.

Regions where the covering parts C are provided are not particularly limited as long as the covering parts C are provided at a plurality of locations with predetermined areas on the surface of the artificial blood vessel VE. In the present embodiment, as shown in FIG. 5, the covering parts C are provided on the portions R21, R31 of the warp yarn 1 that extend so as to cross over a plurality of weft yarns 2 (only the portion R21 that extends so as to cross over a plurality of weft yarns 2 is shown in FIG. 5). More specifically, the covering parts C are provided in parts corresponding to the first portion on the second region side R21 and the first portion on the third region side R31. It should be noted that the covering parts C do not necessarily need to cover all of the plurality of filament yarns provided in the portions that extend so as to cross over a plurality of weft yarns 2 (the first portion on the second region side R21 and the first portion on the third region side R31), and may cover most parts (for example, but not limited to, 50% or more, preferably 80% or more) of the plurality of filament yarns.

As mentioned above, in the portions R21, R31 of the warp yarn 1 which extend so as to cross over a plurality of weft yarns 2, the warp yarn 1 crosses over a plurality of weft yarns 2, so that the artificial blood vessel VE becomes more flexible in the portions R21, R31 than an artificial blood vessel having only a plain weave structure. Moreover, both ends of the portions R21, R31 that extend so as to cross over a plurality of weft yarns 2 become bound by the weft yarn 2 (see the parts P1 and P2 in FIG. 3). In this case, the portions R21, R31 that extend so as to cross over a plurality of weft yarns 2 composed of multifilament yarn, both ends of which are bound, form a three-dimensional structure in which a central part in the extending direction D1 of the warp yarn 1 spreads in the extending direction D2 of the weft yarn 2. As such, the portions R21, R31 that extend so as to cross over a plurality of weft yarns 2 have flexibility and a high blood retaining performance due to the three-dimensional structure, and are further covered by the covering parts C, thereby further improving in blood leakage resistance. Thus, flexibility and blood leakage resistance of the artificial blood vessel VE are further improved.

It should be noted that a structure of the covering part C is not particularly limited as long as it can cover a multifilament yarn in a planar shape. In the present embodiment, the covering part C is composed of a resin layer that is in a state where a multifilament yarn is melted and solidified or that is coated on the surface of the multifilament yarn. The “state where a multifilament yarn is melted and solidified” refers to a state where a part of the multifilament yarn constituting the warp yarn 1 and/or the weft yarn 2 is once melted by heating or the like, and then solidifies to form a planar resin layer. In this case, a plurality of filament yarns in an unmelted state are covered on the surface of the artificial blood vessel VE by planar covering part C that are melted and solidified resin layer. Moreover, the “resin layer coated on the surface” refers to a resin layer formed by coating a resin material in a planar shape on a multifilament yarn constituting the warp yarn 1 and/or the weft yarn 2.

A method of producing an artificial blood vessel VE is not particularly limited, but it can be produced, for example, by the following producing method if the covering part C is composed of a resin layer in a state where a multifilament yarn is in a melted and solidified. First, a base material having a predetermined weaving structure that constitutes the artificial blood vessel VE (for example, see the weaving structure in FIG. 3) is prepared. Next, a heating medium is brought into contact with a surface of the base material on the outer surface (front surface) side of the artificial blood vessel VE, depending on a position where the covering part C is to be provided, before processing the base material into a cylindrical shape. The heated heating medium melts a part of the multifilament yarn on the surface of the base material, which is then cooled and solidified, forming a covering part C of a desired pattern. Next, the base material is processed into a cylindrical shape to produce an artificial blood vessel VE. It should be noted that the heating of the base material with the heating medium may be performed after the base material is processed into a cylindrical shape. During the step of heating the base material with the heating medium (the step of melting the part of the multifilament yarn), the temperature of the heating medium and the heating time, etc. are adjusted. Therefore, the multifilament yarn melts in a thickness direction of the base material at a part thereof on the outer surface side (surface layer part) of the artificial blood vessel VE, but does not melt in the thickness direction on the inner surface side of the artificial blood vessel VE. The part of the multifilament yarn of the base material on the inner surface side of the artificial blood vessel VE remains in a state where a plurality of filament yarns are separated. As a result, an artificial blood vessel VE having a two-layer structure of a covering part C and an inner weaving part IW is obtained. It should be noted that when producing an artificial blood vessel VE having mountain parts M and valley parts V, in addition to the above-described steps, for example, a cylindrical base material is arranged outside a cylindrical core member, and then a wire is wound around from the outside of the artificial blood vessel VE at positions corresponding to positions of the valley parts V, and is heated. As a result, the artificial blood vessel VE having mountain parts M and valley parts V can also be produced. When a resin layer is coated on the surface of the multifilament yarn, a covering part C can be formed on the base material by, instead of the above-mentioned step of heating the base material (the step of melting the part of the multifilament yarn), applying a resin material on the surface of the multifilament yarn in a desired pattern by a publicly-known method. It should be noted that the above-mentioned method of producing the artificial blood vessel VE is merely one example, and the artificial blood vessel VE is not limited to the above-described method of producing the artificial blood vessel VE.

Although the embodiments of the present invention have been described above, the present invention is not limited to the above-described embodiments. The above-described embodiments mainly explain the inventions having the following configurations.
(1) An artificial blood vessel having a predetermined weaving structure in which a warp yarn and a weft yarn are woven,
wherein at least one of the warp yarn and the weft yarn is composed of a multifilament yarn including a plurality of filament yarns, and
wherein the artificial blood vessel has:
a plurality of covering parts provided at a plurality of locations on a surface of the artificial blood vessel, each of the plurality of covering parts covering the multifilament yarn in a planar shape; and
uncovered parts that are provided between the plurality of covering parts and are not covered by the covering parts on the surface of the artificial blood vessel.
(2) The artificial blood vessel of (1), having inner weaving parts in which the plurality of filament yarns of the multifilament yarn extend with being separated from each other on an inner side in a radial direction of the artificial blood vessel with respect to the covering parts.
(3) The artificial blood vessel of (1) or (2), wherein the covering parts and the uncovered parts are alternately provided in an axial direction and/or a circumferential direction of the artificial blood vessel on a part of the surface of the artificial blood vessel.
(4) The artificial blood vessel of any one of (1) to (3), wherein a total area of the plurality of covering parts is 50% to 90%, preferably 60% to 80%, of a surface area of the artificial blood vessel.
(5) The artificial blood vessel of any one of (1) to (4), wherein the covering part is composed of a resin layer that is in a state where the multifilament yarn is melted and solidified or that is coated on the surface of the multifilament yarn.
(6) The artificial blood vessel of any one of (1) to (5),
wherein the warp yarn extends along a length direction of the artificial blood vessel,
wherein the warp yarn is composed of the multifilament yarn,
wherein the warp yarn has a portion that extends so as to cross over a plurality of weft yarns, and
wherein the covering parts are provided on the portion that extends so as to cross over the plurality of weft yarns.
(7) The artificial blood vessel of any one of (1) to (6), wherein mountain parts and valley parts are alternately formed in the axial direction of the artificial blood vessel.

### REFERENCE SIGNS LIST

1, 1a, 1b, 1c, 1d, 1e, 1f, 1g, 1h, 1i, 1j, 1k, 1l. Warp yarn
2, 2a, 2b, 2c, 2d, 2e, 2f, 2g, 2h, 2i, 2j, 2k, 2l. Weft yarn
C. Covering part
D1. Extending direction of warp yarn (axial direction of artificial blood vessel)
D2. Extending direction of weft yarn (circumferential direction of artificial blood vessel)
IW. Inner weaving part
M. Mountain part
Mt. Top part of mountain part
P1. Part of weft yarn that binds both ends of first portion on second region side
P2. Part of weft yarn that binds both ends of first portion on third region side
PL, PL1, PL2. Planar part
R1. First region
R2. Second region
R21. First portion on second region side (portion that extends so as to cross over a plurality of weft yarns)
R22. Second portion on second region side (portion that extends so as to cross over one weft yarn)
R3. Third region
R31. First portion on third region side (portion that extends so as to cross over a plurality of weft yarns)
R32. Second portion on third region side (portion that extends so as to cross over one weft yarn)
UC. Uncovered part
V. Valley part
Vb. Bottom part of valley part
VE. Artificial blood vessel
X. Axis of artificial blood vessel
θ. Angle between planar part on one side and planar part on the other side

1.-7. (canceled)

8. An artificial blood vessel having a predetermined weaving structure in which a warp yarn and a weft yam are woven,
wherein at least one of the warp yarn and the weft yarn is composed of a multifilament yarn including a plurality of filament yarns, and
wherein the artificial blood vessel has:
a plurality of covering parts provided at a plurality of locations on a surface of the artificial blood vessel, each of the plurality of covering parts covering the multifilament yarn in a planar shape; and
uncovered parts that are provided between the plurality of covering parts and are not covered by the covering parts on the surface of the artificial blood vessel.

9. The artificial blood vessel of claim 8, having inner weaving parts in which the plurality of filament yarns of the multifilament yarn extend with being separated from each other on an inner side in a radial direction of the artificial blood vessel with respect to the covering parts.

10. The artificial blood vessel of claim 8, wherein the covering parts and the uncovered parts are alternately provided in an axial direction and/or a circumferential direction of the artificial blood vessel on a part of the surface of the artificial blood vessel.

11. The artificial blood vessel of claim 8, wherein a total area of the plurality of covering parts is 50% to 90% of a surface area of the artificial blood vessel.

12. The artificial blood vessel of claim 8, wherein the covering part is composed of a resin layer that is in a state where the multifilament yarn is melted and solidified or that is coated on the surface of the multifilament yarn.

13. The artificial blood vessel of claim 8, wherein the warp yarn extends along a length direction of the artificial blood vessel,
wherein the warp yarn is composed of the multifilament yarn,
wherein the warp yarn has a portion that extends so as to cross over a plurality of weft yarns, and
wherein the covering parts are provided on the portion that extends so as to cross over the plurality of weft yarns.

14. The artificial blood vessel of claim 8, wherein mountain parts and valley parts are alternately formed in the axial direction of the artificial blood vessel.
